SEQUENCE LISTING (25 sequences)

SEQ ID NO: 1 - DNA, 21 bp, Artificial Sequence
  Stop codon and +4 nucleotide.
tcgacgtgcgattgaccgttc

SEQ ID NO: 2 - DNA, 21 bp, Artificial Sequence
  Stop codon and +4 nucleotide.
tcgacgtgcgattgatcgttc

SEQ ID NO: 3 - DNA, 21 bp, Artificial Sequence
  Stop codon and +4 nucleotide.
tcgacgtgcgattgaacgttc

SEQ ID NO: 4 - DNA, 21 bp, Artificial Sequence
  Stop codon and +4 nucleotide.
tcgacgtgcgattgagcgttc

SEQ ID NO: 5 - DNA, 21 bp, Artificial Sequence
  Stop codon and +4 nucleotide.
tcgacgtgcgattagccgttc

SEQ ID NO: 6 - DNA, 21 bp, Artificial Sequence
  Stop codon and +4 nucleotide.
tcgacgtgcgattagtcgttc

SEQ ID NO: 7 - DNA, 21 bp, Artificial Sequence
  Stop codon and +4 nucleotide.
tcgacgtgcgattagacgttc

SEQ ID NO: 8 - DNA, 21 bp, Artificial Sequence
  Stop codon and +4 nucleotide.
tcgacgtgcgattaggcgttc

SEQ ID NO: 9 - DNA, 21 bp, Artificial Sequence
  Stop codon and +4 nucleotide.
tcgacgtgcgattaaccgttc

SEQ ID NO: 10 - DNA, 21 bp, Artificial Sequence
  Stop codon and +4 nucleotide.
tcgacgtgcgattaatcgttc

SEQ ID NO: 11 - DNA, 21 bp, Artificial Sequence
  Stop codon and +4 nucleotide.
tcgacgtgcgattaaacgttc

SEQ ID NO: 12 - DNA, 21 bp, Artificial Sequence
  Stop codon and +4 nucleotide.
tcgacgtgcgattaagcgttc

SEQ ID NO: 13 - DNA, 21 bp, Artificial Sequence
  Control for SEQ ID NO1.
tcgacgtgcgatcgaccgttc

SEQ ID NO: 14 - DNA, 21 bp, Artificial Sequence
  Control for SEQ ID NO2.
tcgacgtgcgatcgatcgttc

SEQ ID NO: 15 - DNA, 21 bp, Artificial Sequence
  Control for SEQ ID NO3.
tcgacgtgcgatcgaacgttc

SEQ ID NO: 16 - DNA, 21 bp, Artificial Sequence
  Control for SEQ ID NO4.
tcgacgtgcgatcgagcgttc

SEQ ID NO: 17 - DNA, 21 bp, Artificial Sequence
  Control for SEQ ID NO5.
tcgacgtgcgatcagccgttc

SEQ ID NO: 18 - DNA, 21 bp, Artificial Sequence
  Control for SEQ ID NO6.
tcgacgtgcgatcagtcgttc

SEQ ID NO: 19 - DNA, 21 bp, Artificial Sequence
  Control for SEQ ID NO7.
tcgacgtgcgatcagacgttc

SEQ ID NO: 20 - DNA, 21 bp, Artificial Sequence
  Control for SEQ ID NO8.
tcgacgtgcgatcaggcgttc

SEQ ID NO: 21 - DNA, 21 bp, Artificial Sequence
  Control for SEQ ID NO9.
tcgacgtgcgatcaaccgttc

SEQ ID NO: 22 - DNA, 21 bp, Artificial Sequence
  Control for SEQ ID NO10.
tcgacgtgcgatcaatcgttc

SEQ ID NO: 23 - DNA, 21 bp, Artificial Sequence
  Control for SEQ ID NO11.
tcgacgtgcgatcaaacgttc

SEQ ID NO: 24 - DNA, 21 bp, Artificial Sequence
  Control for SEQ ID NO12.
tcgacgtgcgatcaagcgttc

SEQ ID NO: 25 - DNA, 39 bp, Mus musculus
tcgacgtctttgaaagagcaataaaatggcttcaactat

(19) United States
(12) Patent Application Publication
(10) Pub. No.: US 2003/0049666 A1
(43) Pub. Date: Mar. 13, 2003

(54) Tissue culture assay for measuring drug induced translational recoding at premature stop codons and frameshift mutations

(75) Inventors: Michael T. Howard, Salt Lake City, UT (US); Raymond F. Gesteland, Salt Lake City, UT (US); John F. Atkins, Salt Lake City, UT (US); Kevin M. Flanigan, Salt Lake City, UT (US)

Correspondence Address: ALAN J. HOWARTH, P.O. BOX 1909, SANDY, UT, 84091-1909, US

(73) Assignee: University of Utah

(21) Appl. No.: 10/210,200

(22) Filed: Jul. 31, 2002

Related U.S. Application Data
(60) Provisional application No. 60/309,041, filed on Jul. 31, 2001.

(51) Int. Cl. C12Q 1/68; C12Q 1/66

(52) U.S. Cl. 435/6; 435/8

(57) ABSTRACT

Assays for screening small-molecule compounds for their ability to induce translational readthrough of stop codons are disclosed. The assays utilize a dual enzymatic reporter plasmid system, wherein one reporter acts as an internal standard and the second reporter measures the translational recoding event induced by the small-molecules. The genetic sequence mutations of interest are placed on the plasmid between the two reporter genes and the plasmids are transfected into tissue culture cells. The cells are then grown in the presence of varying amounts of small-molecule compounds and the induction of translational readthrough is measured.

### CROSS-REFERENCE TO RELATED APPLICATIONS

This application claims the benefit of U.S. Provisional Application No. 60/309,041, filed Jul. 31, 2001, which is hereby incorporated by reference in its entirety.

### STATEMENT REGARDING FEDERALLY SPONSORED RESEARCH OR DEVELOPMENT

This invention was made with government support under Department of Energy grant DEFG03-99ER62732 and National Institutes of Health grant RO1-GM48152-05. The government has certain rights in the invention.

### BACKGROUND OF THE INVENTION

This invention relates to an assay for testing the ability of small-molecule compounds to induce translational recoding at premature stop codons and frameshift mutations. More particularly, this invention relates to the use of a dual enzymatic reporter system in which the ability of small-molecule compounds to induce translational readthrough of stop codons, frameshifting, or translational bypass at sites of genetic sequence mutation can be readily assayed in tissue culture cells.

A subset of common genetic diseases arises from point mutations that introduce premature stop codons into important coding sequences. Among these is Duchenne muscular dystrophy (DMD) in which approximately 5 to 10% of cases are due to a premature stop codon mutation in the coding sequence. T. W. Prior et al., Spectrum of small mutations in the dystrophin coding region, 57 Am. J. Hum. Genet. 22-33 (1995). The aminoglycoside antibiotics have recently been suggested as possible therapeutic interventions for treating patients who carry such mutations, A. S. Mankin & S. W. Liebman, Baby, don't stop!, 23 Nat. Genet.8-10 (1999), as a result of the ability of these antibiotics to cause translational readthrough of stop codons. J. M. Wilhelm et al., Aminoglycoside antibiotics and eukaryotic protein synthesis: structure-function relationships in the stimulation of misreading with a wheat embryo system, 17 Biochemistry 1143-1149 (1978); M. I. Recht et al., Basis for prokaryotic specificity of action of aminoglycoside antibiotics, 18 EMBO J. 3133-3138 (1999); A. Singh et al., Phenotypic suppression and misreading Saccharomyces cerevisiae, 277 Nature 146-148 (1979); E. Palmer et al., Phenotypic suppression of nonsense mutants in yeast by aminoglycoside antibiotics, 277 Nature 148-150 (1979); J. F. Burke & A. E. Mogg, Suppression of a nonsense mutation in mammalian cells in vivo by the aminoglycoside antibiotics G-418 and paromomycin, 13 Nucleic Acids Res. 6265-6272 (1985); R. Martin et al., Aminoglycoside suppression at UAG, UAA and UGA codons in Escherichia ia coli and human tissue culture cells, 217 Mol. Gen. Genet.411-418 (1989). This concept was recently demonstrated in an in vivo mouse model for DMD. In that study, it was shown that gentamicin can partially reverse the effects of a premature stop codon in the dystrophin gene of the mdx mouse, resulting in the presence of dystrophin protein in the cell membrane and protection against muscular injury. E. R. Barton-Davis et al., Aminoglycoside antibiotics restore dystrophin function to skeletal muscles of mdx mice, 104 J. Clin. Invest. 375-381 (1999). These results suggest that in a subset of patients with DMD, partial expression of the full-length protein at levels sufficient to alleviate disease symptoms may be induced by aminoglycoside treatment.

It is provocative to think that aminoglycoside treatment has the potential to treat not only a subset of DMD patients, but also a wide range of genetic diseases originating from a nonsense mutation. In addition to the mdx study mentioned previously, G-418 and gentamicin have been shown to restore the expression of the cystic fibrosis transmembrane conductance regulator (CFTR) protein in a bronchial cell line carrying a nonsense mutation in the CFTR gene. D. M. Bedwell et al., Suppression of CFTR premature stop mutation in a bronchial epithelial cell line, 3 Nat. Med. 1280-1284 (1997); M. Howard et al., Aminoglycoside antibiotics restore CFTR function by overcoming premature stop mutations, 2 Nat. Med. 467-469 (1996). It remains to be seen whether all nonsense mutations respond equally to aminoglycoside treatments. Earlier studies on aminoglycoside-induced stop codon readthrough indicate that the efficiency of readthrough may be influenced by the sequence context in which the stop codon occurs. In one study, the stop codons located at various positions within the chloramphenicol acetyl transferase gene were tested in human tissue culture cells and found to have different levels of aminoglycoside induced stop codon readthrough. R. Martin et al., Codon context effects on nonsense suppression in human cells, 21 Biochem. Soc. Trans. 846-851 (1993). In these studies, the 5′ and 3′ sequence context varied for each stop codon such that it was not possible to determine whether sequences upstream or downstream or the identity of the stop codon itself was responsible for variations in readthrough levels. This sequence context effect was subsequently addressed by testing aminoglycoside-induced readthrough of the UAG stop codon with a fixed 5′ sequence context and varying only the downstream 3′ nucleotide. The nucleotide located immediately downstream of the stop codon was shown to influence aminoglycoside-induced readthrough; however, UGA and UAA stop codons were not tested in these more systematic experiments. In light of these results, it seems likely that aminoglycoside treatment results in variable levels of stop codon readthrough depending on the identity of the nonsense codon and its sequence context.

In the absence of aminoglycosides, the efficiency of release factor recognition and translational termination is influenced by the sequence context in which the stop codon is embedded. R. Martin et al., Codon context effects on nonsense suppression in human cells, 21 Biochem. Soc. Trans. 846-851 (1993); W. P. Tate & S. A. Mannering, Three, four or more: the translational stop signal at length, 21 Mol. Microbiol. 213-219 (1996); B. Bonetti et al., The efficiency of translation termination is determined by a synergistic interplay between upstream and downstream sequences in Saccharomyces cerevisiae, 251 J. Mol. Biol. 334-345 (1995). Termination efficiency is determined by the identity of the stop codon (UGA >UAG >UAA) and sequences located 5′, A. L. Arkov et al., 5′ contexts of Escherichia coli and human termination codons are similar, 23 Nucleic Acids Res. 4712-4716 (1995); S. Mottagui-Tabar et al., The influence of 5′ codon context on translation termination in Saccharomyces cerevisiae, 257 Eur. J. Biochem. 249-254 (1998), and 3′ of the stop codon, although the strongest sequence context effect comes from the nucleotide in the position immediately after the stop codon (+4). W. P. Tate & S. A. Mannering, Three, four or more: the translational stop signal at length, 21 Mol. Microbiol. 213-219 (1996); C. M. Brown et al., Sequence analysis suggests that tetra-nucleotides signal the termination of protein synthesis in eukaryotes, 18 Nucleic Acids Res. 6339-6345 (1990); K. K. McCaughan et al., Translational termination efficiency in mammals is influenced by the base following the stop codon, 92 Proc. Nat'l Acad. Sci. U.S.A. 5431-5435 (1995). For this position, purines create a more effective termination environment than pyrimidines. In addition, analysis of highly expressed genes indicates a strong bias at the +4 position. Approximately 90% of the most highly expressed genes in mammals carry a purine in this position. W. P. Tate & S. A. Mannering, Three, four or more: the translational stop signal at length, 21 Mol. Microbiol. 213-219 (1996). It would be expected that efficient termination would compete with aminoglycoside-induced readthrough of stop codons.

In some cases, the sequence context effect can be quite dramatic, resulting in translational readthrough of premature stop codon mutations at a high enough frequency to slow disease progression in the absence of any drug treatments. For example, UGA nonsense mutations located about one third of the way through the human CFTR gene cause less severe pulmonary, problems than some other missense mutations, B. S. Kerem et al., Identification of mutations in regions corresponding to the two putative nucleotide (ATP)-binding folds of the cystic fibrosis gene, 87 Proc. Nat'l Acad. Sci. U.S.A. 8447-8451 (1990); H. Cuppens et al., A child, homozygous for a stop codon in exon 11, shows milder cystic fibrosis symptoms than her heterozygous nephew, 27 J. Med. Genet. 717-719 (1990); G. R. Cutting et al., Two patients with cystic fibrosis, nonsense mutations in each cystic fibrosis gene, and mild pulmonary disease, 323 N. Engl. J. Med. 1685-1689 (1990); G. R. Cutting et al., A cluster of cystic fibrosis mutations in the first nucleotide-binding fold of the cystic fibrosis conductance regulator protein, 346 Nature 366-369 (1990), and studies of mutations at the equivalent position in the homologous yeast gene revealed that one stop codon was read through with an efficiency of approximately 10%. K. Fearon et al., Premature translation termination mutations are efficiently suppressed in a highly conserved region of yeast Ste6p, a member of the ATP-binding cassette (ABC) transporter family, 269 J. Biol. Chem. 17802-17808 (1994).

The same factors that determine translational termination efficiency may also influence aminoglycoside-induced readthrough of premature stop codons. Understanding how each of the stop codons and the surrounding sequence context affect the efficiency of aminoglycoside-induced readthrough has direct implications for potential aminoglycoside therapy of DMD and other genetic diseases. Such information may allow for an accurate prediction of a patient's response to a given aminoglycoside treatment or even the ability to tailor treatment regimens for specific nonsense mutations in specific contexts. For example, a patient with a nonsense mutation in a context that allows for high readthrough efficiencies when exposed to aminoglycosides may require a lower concentration of drug or less frequent treatments, thus avoiding or attenuating the toxicity often associated with aminoglycoside therapy, whereas a patient carrying a nonsense mutation in a less favored context may require a more aggressive treatment regimen. Finally, understanding these sequence-specific effects may influence interpretation of treatment trials in DMD and other genetic diseases, and future trials may benefit from stratifying patients according to sequence-specific readthrough efficiencies as determined by in vitro assays.

In view of the foregoing, it will be appreciated that providing a rapid and quantitative approach to measuring both the efficiency of drug-induced translational readthrough of nonsense mutations and how that efficiency is influenced by the sequence context of the stop codon using a dual reporter system would be a significant advancement in the art.

### BRIEF SUMMARY OF THE INVENTION

It is an advantage of the present invention to provide a tissue culture assay for the identification and efficient screening of small-molecule compounds for the ability to induce translational recoding events.

It is another advantage of the invention to provide a tissue culture assay for analyzing a given genetic mutation for induction by small-molecule compounds of the desired translational recoding event.

It is also an advantage of the invention to provide a tissue culture assay for analyzing a given genetic mutation for induction by small-molecule compounds of the desired translational recoding event in a given cell type.

It is still another advantage of the invention to provide a tissue culture assay for optimizing a drug treatment regime for a given genetic mutation.

These and other advantages can be achieved by utilizing a rapid and quantitative approach to measuring both the efficiency of drug-induced translational readthrough of nonsense mutations and how that efficiency is influenced by the sequence context of the stop codon using a dual reporter system.

An illustrative method of screening small-molecule compounds for ability to induce translational readthrough of a stop codon according to the present invention comprises:

(a) providing a translational reporter vector comprising a cloning site disposed between a first coding sequence encoding a first reporter and a second coding sequence encoding a second reporter and inserting a test DNA comprising an in-frame stop codon at the cloning site to form a test vector such that the first and second coding sequences are in a same reading frame but separated by the in-frame stop codon;

(b) inserting a control DNA in the translational reporter vector at the cloning site to form a control vector such that the first and second coding sequences are in the same reading frame and are not separated by an in-frame stop codon;

(c) separately transfecting aliquots of cells with the test vector and the control vector to result in transfected cells containing the test vector and transfected cells containing the control vector;

(d) separately incubating the transfected cells containing the test vector and the transfected cells containing the control vector in both the presence and absence of a small-molecule compound under conditions suitable for expression of the first reporter and, if translational readthrough of the stop codon occurs, expression of the second reporter;

(e) lysing the incubated cells and determining activities of the first reporter and of the second report in both the presence and absence of the small-molecule compound; and

(f) calculating ratios of second reporter activity to first reporter activity in both the presence and absence of the small-molecule compound, comparing such ratios, and determining that the small-molecule compound has induced translational readthrough of the stop codon when the ratio of second reporter activity to first reporter activity in the presence of the small-molecule compound exceeds the ratio of second reporter activity to first reporter activity in the absence of the small-molecule compound.

An illustrative method of screening drugs for potential for treating a genetic disease that is treatable by inducing translational readthrough of a stop codon causally linked with the genetic disease according to the present invention comprises:

(a) providing a translational reporter vector comprising a cloning site disposed between a first coding sequence encoding a first reporter and a second coding sequence encoding a second reporter and inserting a test DNA comprising the stop codon causally linked with the genetic disease and flanking sequences thereof in the cloning site to form a test vector such that the first and second coding sequences are in a same reading frame but separated by the stop codon causally linked with the genetic disease, wherein such stop codon is in the same reading frame as the first and second coding sequences;

(b) inserting a control DNA in the translational reporter vector at the cloning site to form a control vector such that the first and second coding sequences are in the same reading frame and are not separated by a stop codon in the same reading frame as the first and second coding sequences;

(c) separately transfecting aliquots of cells with the test vector and the control vector to result in transfected cells containing the test vector and transfected cells containing the control vector;

(d) separately incubating the transfected cells containing the test vector and the transfected cells containing the control vector in both the presence and absence of a drug under conditions suitable for expression of the first reporter and, if translational readthrough of the stop codon occurs, expression of the second reporter;

(e) lysing the incubated cells and determining activities of the first reporter and of the second report in both the presence and absence of the drug; and

(f) calculating ratios of activity of the second reporter to activity of the first reporter both in the presence and the absence of the drug, comparing such ratios, and determining that the drug has induced translational readthrough of the stop codon, and thereby exhibits potential for treating the genetic disease, when the ratio of activity of the second reporter to activity of the first reporter in the presence of the drug exceeds the ratio of activity of the second reporter to activity of the first reporter in the absence of the drug.

An illustrative method of screening drugs for potential for treating Duchenne muscular dystrophy caused by premature translational termination of dystrophin caused by a mutation that introduces a premature stop codon into a coding sequence of dystrophin according to the present invention comprises:

(a) providing a translational reporter vector comprising a cloning site disposed between a first coding sequence encoding a first reporter and a second coding sequence encoding a second reporter and inserting a test DNA comprising at least a portion the coding sequence of dystrophin comprising the premature stop codon to form a test vector such that the first and second coding sequences are in a same reading frame but are separated by the premature stop codon, wherein such premature stop codon is in the same reading frame as the first and second coding sequences;

(b) inserting a control DNA in the translational reporter vector at the cloning site to form a control vector such that the first and second coding sequences are in the same reading frame and are not separated by a stop codon in such reading frame;

(c) separately transfecting aliquots of cells with the test vector and the control vector to result in transfected cells containing the test vector and transfected cells containing the control vector;

(d) separately incubating the transfected cells containing the test vector and the transfected cells containing the control vector in both the presence and absence of a drug under conditions suitable for expression of the first reporter and, if translational readthrough of the premature stop codon occurs, expression of the second reporter;

(e) lysing the incubated cells and determining activities of the first reporter and of the second reporter both in the presence and in the absence of the drug; and

(f) calculating ratios of the activity of the second reporter to the activity of the first reporter both in the presence and in the absence of the drug, comparing such ratios, and determining that the drug has induced translational readthrough of the premature stop codon, and thereby exhibits potential for treating Duchenne muscular dystrophy, when the ratio of activity of the second reporter to the activity of the first reporter in the presence of the drug exceeds the ratio of the activity of the second reporter to the activity of the first reporter in the absence of the drug.

An illustrative method for predicting whether a patient afflicted with a genetic disease caused by a mutation creating a premature stop codon will be effectively treated for such genetic disease with a selected drug according to the present invention comprises:

(a) providing a translational reporter vector comprising a cloning site disposed between a first coding sequence encoding a first reporter and a second coding sequence encoding a second reporter and inserting a test DNA derived from the patient comprising the premature stop codon and flanking sequences thereof in the cloning site to form a test vector such that the first and second coding sequences are in a same reading frame but separated by the premature stop codon in such same reading frame;

(b) inserting a control DNA in the translational reporter vector at the cloning site to form a control vector such that the first and second coding sequences are in the same reading frame and are not separated by a stop codon in the same reading frame as the first and second coding sequences;

(c) separately transfecting aliquots of cells with the test vector and the control vector to result in transfected cells containing the test vector and transfected cells containing the control vector;

(d) separately incubating the transfected cells containing the test vector and the transfected cells containing the control vector in both the presence and absence of the selected drug under conditions suitable for expression of the first reporter and, if translational readthrough of the premature stop codon occurs, expression of the second reporter;

(e) lysing the incubated cells and determining activities of the first reporter and of the second report in both the presence and absence of the selected drug; and

(f) calculating ratios of activity of the second reporter to activity of the first reporter both in the presence and the absence of the selected drug, comparing such ratios, and determining that the selected drug has induced translational readthrough of the premature stop codon, and thereby the patient can be effectively treated for such genetic diseases, when the ratio of activity of the second reporter to activity of the first reporter in the presence of the selected drug exceeds the ratio of activity of the second reporter to activity of the first reporter in the absence of the selected drug.

An illustrative method for screening drugs for ability to induce translational recoding at frameshift mutations according to the present invention comprises:

(a) providing a translational reporter vector comprising a cloning site disposed between a first coding sequence encoding a first reporter and a second coding sequence encoding a second reporter wherein the first and second coding sequences are in different reading frames and inserting a test DNA in the translational reporter vector at the cloning site to form a test vector such that the first and second coding sequences remain in different reading frames;

(b) inserting the test DNA in the translation reporter vector at the cloning site to form a control vector such that the first and second coding sequences are in a same reading frame;

(c) separately transfecting aliquots of cells with the test vector and the control vector to result in transfected cells containing the test vector and transfected cells containing the control vector;

(d) incubating the transfected cells containing the test vector and the transfected cells containing the control vector in the presence and in the absence of a selected drug under conditions suitable for expression of the first reporter and, if translational recoding of the frameshift mutation occurs, expression of the second reporter;

(e) lysing the transfected cells containing the test vector and the transfected cells containing the control vector and determining activities of the first reporter and the second reporter both in the presence and in the absence of the selected drug; and

(f) calculating ratios of the activity of the second reporter to the activity of the first reporter both in the presence and in the absence of the drug, comparing such ratios, and determining that the drug has induced translational recoding of the frameshift mutation when the ratio of activity of the second reporter to the activity of the first reporter in the presence of the selected drug exceeds the ratio of the activity of the second reporter to the activity of the first reporter in the absence of the selected drug.

An illustrative method for predicting whether a patient afflicted with a genetic disease caused by a mutation creating a frameshift will be effectively treated for such genetic disease with a selected drug according to the present invention comprises:

(a) providing a translational reporter vector comprising a cloning site disposed between a first coding sequence encoding a first reporter and a second coding sequence encoding a second reporter and inserting a test DNA derived from the patient comprising the frameshift and flanking sequences thereof in the cloning site to form a test vector such that the first and second coding sequences are in different reading frames;

(b) inserting the test DNA in the translational reporter vector at the cloning site to form a control vector such that the first and second coding sequences are in a same reading frame;

(c) separately transfecting aliquots of cells with the test vector and the control vector to result in transfected cells containing the test vector and transfected cells containing the control vector;

(d) separately incubating the transfected cells containing the test vector and the transfected cells containing the control vector in both the presence and absence of the selected drug under conditions suitable for expression of the first reporter and, if translational recoding of the frameshift occurs, expression of the second reporter;

(e) lysing the incubated cells and determining activities of the first reporter and of the second report in both the presence and absence of the selected drug; and

(f) calculating ratios of activity of the second reporter to activity of the first reporter both in the presence and the absence of the selected drug, comparing such ratios, and determining that the selected drug has induced recoding of the frameshift, and thereby the patient can be effectively treated for such genetic diseases, when the ratio of activity of the second reporter to activity of the first reporter in the presence of the selected drug exceeds the ratio of activity of the second reporter to activity of the first reporter in the absence of the selected drug.

### BRIEF DESCRIPTION OF THE SEVERAL VIEWS OF THE DRAWINGS

FIG. 1 shows a map of plasmid p2luc wherein rluc indicates a renilla (sea pansy) luciferase coding sequence;fluc indicates a firefly luciferase coding sequence; Amp^{r} indicates an ampicillin resistance coding sequence; and ori indicates an origin of replication functional in Escherichia coli.

FIGS. 2A-I show stop codon readthrough induced by the aminoglycosides G-418 (FIGS. 2A-C), gentamicin (FIGS. 2D-F), and paromomycin (FIGS. 2G-I) in p2luc plasmids containing sequences encoding the stop codons UGA N (FIGS. 2A, 2D & 2G), UAG N (FIGS. 2B, 2E & 2H), and UAA N (FIGS. 2C, 2F & 2I), where N is either A (filled triangles), C (squares), G (circles), or U (open triangles), in HEK293 cells; the efficiency of stop codon readthrough is shown on the y axis, and the concentration in milligrams per milliliter of aminoglycoside added to the growth medium is shown on the x axis.

### DETAILED DESCRIPTION

Before the present tissue culture assay is disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to incubating cells in the presence of “a drug” includes reference to incubating the cells in the presence of a mixture of two or more drugs, and reference to “a cell” includes reference to one or more of such cells.

In describing and claiming the present invention, the following terminology will be used in accordance with the definitions set out below.

As used herein, “vector” means a plasmid, virus, or the like that is used for carrying DNA into a cell.

As used herein, “recoding” refers to a phenomenon wherein the rules for translation decoding are temporarily altered through specific sites and signals built into the mRNA sequences. I. Brierly, Ribosomal frameshifting on viral RNAs, 76 J. Gen. Virol. 1885-1892 (1995); R. F. Gesteland & J. Atkins, Recoding: dynamic reprogramming of translation, 65 Annu. Rev. Biochem. 741-768 (1996). In mammalian cells, three kinds of recoding have been described. First, redefinition of stop codons to sense codons (i.e., readthrough) allows synthesis of selenocysteine-containing proteins, A. Bock et al., Selenoprotein synthesis: an expansion of the genetic code, 16 Trends Biochem. Sci. 463-467 (1991); S. C. Low & M. J. Berry, Knowing when not to stop: selenocysteine incorporation in eukaryotes, 21 Trends Biochem. Sci. 203-208 (1996), and synthesis of elongated proteins in many RNA viruses, such as Moloney murine leukemia virus (MuLV), Y. Yoshinaka et al., Murine leukemia virus protease is encoded by the gag-pol gene and is synthesized through suppression of an amber termination codon, 82 Proc. Nat'l Acad. Sci. U.S.A. 1618-1622 (1985). Second, +1 frameshifting regulates expression of ornithine decarboxylase antizyme. The system is autoregulatory and depends on the concentration of polyamines. S. Hayashi et al., Omithine decarboxylase antizyme: a novel type of regulatory protein, 21 Trends Biochem. Sci. 27-30 (1996). Third, -1 frameshifting is used to synthesize the GagPol precursor polyprotein in retroviruses (except spumaretroviruses) that have gag, (pro), and pol genes in different reading frames, J. Enssle et al., Foamy virus reverse transcriptase is expressed independently from the gag protein, 93 Proc. Nat'l Acad. Sci. U.S.A. 4137-4141 (1996). Examples are the mouse mammary tumor virus (MMTV) gag-pro frameshift, T. Jacks et al., Two efficient ribosomal frameshifting events are required for synthesis of mouse mammary tumor virus gag-related polyproteins, 84 Proc. Nat'l Acad. Sci. U.S.A. 4298-4302 (1987); R. Moore et al., Complete nucleotide sequence of a milk-transmitted mouse mammary tumor virus: two frameshift suppression events are required for translation of gag and pol, 61 J. Virol. 480-490 (1987), and the human immunodeficiency virus type 1 (HIV-1) gag-pol frameshift, N. T. Parkin et al., Human immunodeficiency virus type 1 gag-pol frameshifting is dependent on downstream mRNA secondary structure: demonstration by expression in vivo, 66 J. Virol. 5147-5151 (1992).

As used herein, “aminoglycoside” means any of the well known class of polycationic compounds characterized by amino sugars in glycosidic linkages including, for example, streptomycin, gentamicin, tobramycin, kanamycin, neomycin, paromomycin, G-418, and the like, and mixtures thereof.

As used herein, a “small-molecule compound” means a compound having a molecular weight of less than about 1000.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

A rapid and quantitative assay for measuring the efficiency of drug-induced translational readthrough of nonsense mutations and how that efficiency is influenced by the sequence context of the stop codons in tissue culture has been designed. The present invention discloses novel assays that can use, for example, a dual-luciferase reporter system disclosed in U.S. Pat. No. 6,143,502. In addition, the nonsense mutation and flanking sequences from the mdx mouse dystrophin gene were tested for gentamicin-induced readthrough in a mouse myoblast cell line.

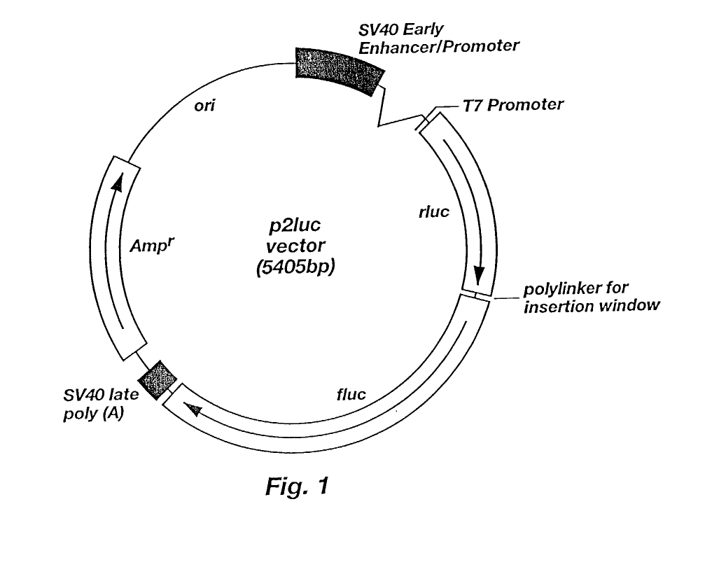

FIG. 1 shows the plasmid, p2luc, which comprises an illustrative dual luciferase reporter system for measuring recoding efficiencies in vivo or in vitro from a single construct. As described in U.S. Pat. No. 6,143,502, the firefly luciferase gene (fluc) was cloned downstream of the renilla luciferase gene (rluc) into an altered vector pRL-SV40 vector. Expression features for initiation and termination of transcription and translation, as well as the nature of the two reporter coding sequences (short enough to be efficiently synthesized in an in vitro translation system), allow application of the same reporter construct for in vivo and in vitro applications. Between the 5′ reporter (rluc) and the 3′ reporter (fluc), a polylinker or multiple cloning site was inserted. The p2luc polylinker has restriction sites for digestion with SalI, BamHI, and SacI restriction endonucleases. The assay using this reporter plasmid combines rapidity of the reactions with very low background levels and provides a powerful assay. In vitro experiments can be performed in 96-well microtiter plates, and in vivo experiments can be performed in 6-well culture dishes, for example. This makes the dual-luciferase assay suitable for high throughput screening approaches.

The dual-luciferase assay is designed such that synthesis of the second reporter (firefly luciferase) is dependent on recoding. On its own, however, the amount of this reporter is not a direct reflection of the efficiency of recoding. In the absence of in-frame stop codons, a significant proportion of translating ribosomes disengage prematurely from the mRNA, which is often referred to as ribosome drop off. Results from several laboratories have shown that 50% or more of Escherichia coli ribosomes drop off during synthesis of p-galactosidase. J. L. Manley, Synthesis and degradation of termination and premature-termination fragments of β-galactosidase in vitro and in vivo, 125 J. Mol. Biol. 407-432 (1978); C. G. Kurland et al., Limitations of translational accuracy, in F. C. Neidhardt et al., Escherichia coli and Salmonella typhimurium. Cellular and Molecular Biology 979-1004 (2^{d} ed 1996). Ribosomes that drop off while decoding the firefly reporter will lead to an underestimate of the proportion of ribosomes that respond to the recoding signals unless a correction is made. The basis for a correction factor is the assumption that drop off during synthesis of the firefly reporter is proportional to completion of synthesis of this reporter. The correction factor is provided by a control in which all ribosomes that complete synthesis of the first reporter (renilla luciferase) continue translation by starting synthesis of the firefly luciferase reporter.

The fate of ribosomes is assessed by the level of their products. This can be expressed by the equation p=RF/(RF+Rf), where p is the proportion of ribosomes that respond to the recoding signal that complete synthesis of the firefly reporter; Rf represents the products of translating ribosomes that responded to the recoding signal but aborted before completing synthesis of the firefly reporter; and RF represents products exhibiting firefly luciferase activity (their synthesis requires complete translation of the coding regions for both reporters).

Because the test sequence and its corresponding control are identical downstream of the recoding signal, it is assumed that the proportion, p, is the same for both constructs:

p=Rₜₑₛₜ/(Rₜₑₛₜ+Rfₜₑₛₜ)=RF_{control}/(RF_{control}+Rf_{control})

Recoding efficiency can be expressed by the number of ribosomes that respond to the recoding signal divided by the number of ribosomes that reach the recoding signal (i.e., that have completed translation of the renilla reporter):

Recoding efficiency=(RFₜₑₛₜ+Rfₜₑₛₜ)/(RFₜₑₛₜ+Rfₜₑₛₜ+Rₜₑₛₜ+Rₜₑₛₜ)

[(RFₜₑₛₜ)/(RFₜₑₛₜ+Rfₜₑₛₜ+Rₜₑₛₜ)]/P

where R is the product of translation of the renilla reporter coding sequence, with ribosomes terminating at the zero frame terminator located at or within a short distance 3′ to the site of recoding.

The measured firefly luciferase activity (Fa) is given by the number of peptides that have firefly luciferase activity (RF) multiplied by the specific activity of these peptides (Φ). Because the peptide sequences of a test sequence and its control are identical, the specific activity of molecules synthesized from the test and its control reaction are equal:

Faₜₑₛₜ=RFₜₑₛₜ×Φ and Fa_{control}=RF_{control}×Φ

The measured renilla luciferase activity (Ra) is given by the number of peptides that have renilla luciferase activity multiplied by the specific activity (Ω) of the respective species:

Raₜₑₛₜ=Rₜₑₛₜ×Ω_{R}+Rfₜₑₛₜ×Ω_{Rf}+RFₜₑₛₜ×Ω_{RF} and

Ra_{control}=Rf_{control}×Ω_{Rf}+RF_{control})×Ω

Experimentally, the specific activity of renilla luciferase was not altered by C-terminal extensions of the different constructs (see below). Then:

Raₜₑₛₜ=(Rₜₑₛₜ+Rfₜₑₛₜ+RFₜₑₛₜ)×Ω and

Ra_{control}=(Rf_{control}+RF_{control})×Ω

The experimentally established value for the ratio of firefly over renilla luciferase activity for the test sequence can be described as:

(Faₜₑₛₜ/Raₜₑₛₜ)=[RFₜₑₛₜ/(Rₜₑₛₜ+Rfₜₑₛₜ+RFₜₑₛₜ)]×[Φ/Ω]

and the luciferase activity ratio of the control construct as:

(Fa_{control}/Ra_{control})=[RF_{control}/(Rf_{control}+RF_{control})]×[Φ/Ω]

It follows that:

Recoding efficiency=[(RF_{tcst})/(RFₜₑₛₜ+Rfₜₑₛₜ+Rₜₑₛₜ)]/P

=(Faₜₑₛₜ/Raₜₑₛₜ)/(Fa_{control}/Ra_{control})

In other words, the activity ratio of the control construct can be used to normalize the activity ratio obtained from the test sequence for drop off occurring downstream from the recoding signal.

### EXAMPLE 1

The p2luc dual luciferase reporter plasmid system, as described in U.S. Pat. No. 6,143,502, was used to measure the effect of aminoglycosides on stop codon readthrough in tissue culture cells. In this system, the renilla (sea pansy) luciferase and firefly luciferase reporter coding sequences are located on either side of a stop codon (FIG. 1). Stop codon and control constructs (containing a sense codon in place of the stop codon) are transfected in parallel in tissue culture cells. Expression of the upstream renilla luciferase gene provides a way to normalize for differences in transfection efficiencies, translation initiation, and mRNA levels between transfected cultures. Normalization is particularly important when comparing results obtained from constructs containing nonsense mutations, because messages containing premature stop codons have been shown to be preferentially degraded by the nonsense-mediated decay pathway. M. R. Culbertson, RNA surveillance. Unforeseen consequences for gene expression, inherited generic disorders and cancer, 15 Trends Genet. 74-80 (1999). The ability of the dual reporter system to control for differences in mRNA levels between normal and nonsense-containing sequences provides a distinct advantage compared with single reporter or direct protein analysis. After normalization, the differences in downstream firefly luciferase activities between cultures transfected with the stop codon and control sense codon constructs reflect the frequency of stop codon readthrough.

The stop codons of SEQ ID NO: 1 through SEQ ID NO: 12 and SEQ ID NO: 25 were cloned between the two luciferase genes of p2luc, and control reporter plasmids (SEQ ID NO: 13-24) were produced by changing the first nucleotide of the stop codon to a C. More particularly, complimentary oligonucleotides corresponding to the sequences SEQ ID NO: 1 through SEQ ID NO: 25 were synthesized on an Applied Biosystems model 380C synthesizer (Foster City, Calif.) such that when annealed, they would have SalI- and BamHI-compatible ends. The oligonucleotides were ligated in SalI- and BamHI-digested p2luc vector according to methods well known in the art and transformed to Escherichia coli strain SU1675. DNA sequences were verified by autothermocycler sequencing, and plasmids were purified using the Qiagen Midiprep Kit (Valencia, Calif.) according to the manufacturer's specifications.

The oligonucleotides corresponding to SEQ ID NO: 1 through SEQ ID NO: 12 were identical except for nucleotides 13-16, which contained in-frame stop codons followed by a C, T, A, or G nucleotide. The oligonucleotides corresponding to SEQ ID NO: 13 through SEQ ID NO: 24 were the respective controls, which were identical to the oligonucleotides corresponding to SEQ ID NO: 1 through SEQ ID NO: 12 except the first nucleotide of the stop codon was replaced with a C nucleotide, thus changing the stop codon to a sense codon. The oligonucleotide corresponding to SEQ ID NO: 25 comprises the sequence surrounding the mdx mouse dystrophin mutation.

Translational readthrough of the stop codons of oligonucleotides corresponding to SEQ ID NO: 1 through SEQ ID NO: 12 was determined in a human embryonic kidney cell line (HEK293, e.g., ATCC CRL-1573) grown in the presence of varying amounts of G-418, gentamicin, and paromomycin, which have been particularly effective in inducing stop codon readthrough in yeast. A. Singh et al., Phenotypic suppression and misreading Saccharomyces cerevisiae, 277 Nature 146-148 (1979); E. Palmer et al., Phenotypic suppression of nonsense mutants in yeast by aminoglycoside antibiotics, 277 Nature 148-150 (1979). HEK293 cells were maintained as monolayer cultures growing in Dulbecco's Modified Eagle Medium with 1,000 mg/L of D-glucose, L-glutamine, pyridoxine hydrochloride, and 110 mg/L of sodium pyruvate supplemented with 10% fetal bovine serum and 50 units/ml penicillin/50 pg/ml streptomycin. All cells were incubated at 37° C. in an atmosphere of 5% CO₂. All media, penicillin, and streptomycin were obtained from LifeTechnologies (Rockville, Md.); aminoglycosides were purchased from Sigma (St Louis, Mo.); and all sera were obtained from HyClone (Logan, Utah).

Transfections were performed using lipofectamine reagent (LifeTechnologies). HEK293 cells (0.15×10⁵)were plated in 0.34-cm² wells and grown for 24 to 48 hours according to methods well known in the art. All cells were transfected with 0.3 μl of lipofectin and 0.075 μg of plasmid DNA for 15 hours, in serum-free media. Fresh media with serum and varying levels of aminoglycosides (Sigma) were then added, and incubation continued for 24 hours.

Cells were lysed using passive lysis buffer (Promega, Madison, Wis., and both renilla and firefly luciferase activities were determined using the dual luciferase reporter assay (Promega) on a Dynatech MLX Microtiter Plate Luminometer (Burlington, Mass.). For all reactions, light emission was measured for 2 to 12 seconds after 100μl of luminescence substrate was injected. Stop codon readthrough was calculated by comparing the ratio of firefly to renilla luciferase activity in cultures transfected with p2luc stop codon constructs and compared with ratios obtained from cultures transfected with p2luc control constructs.

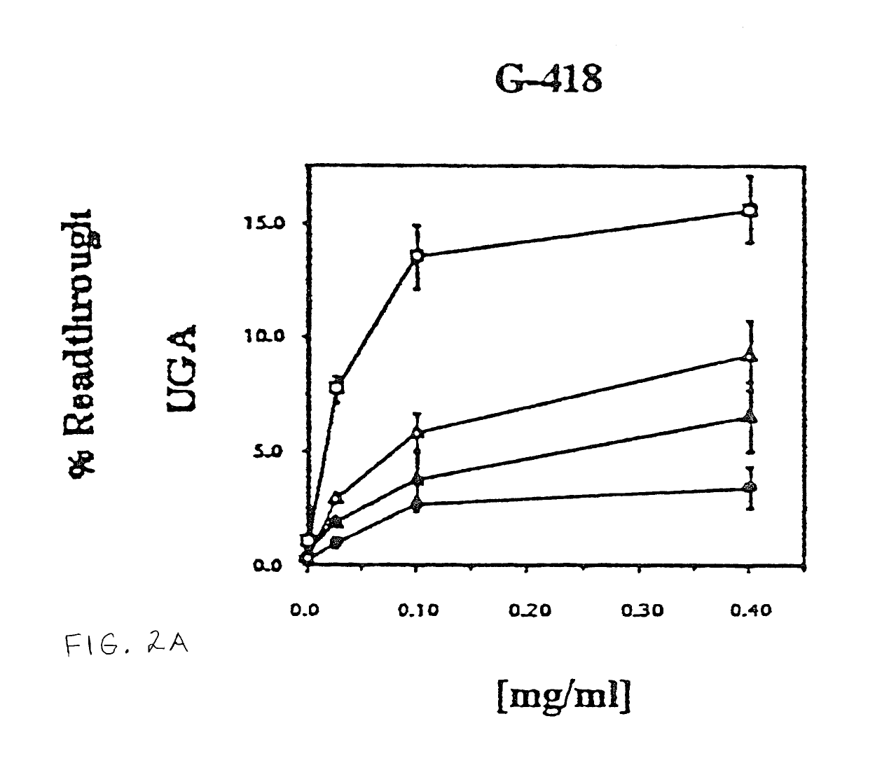

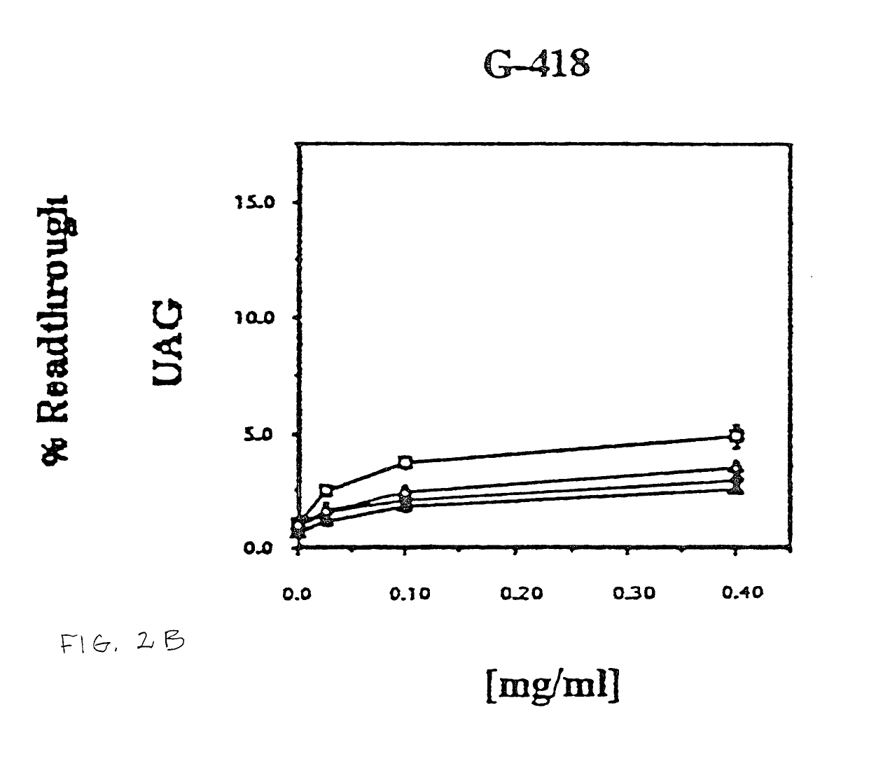

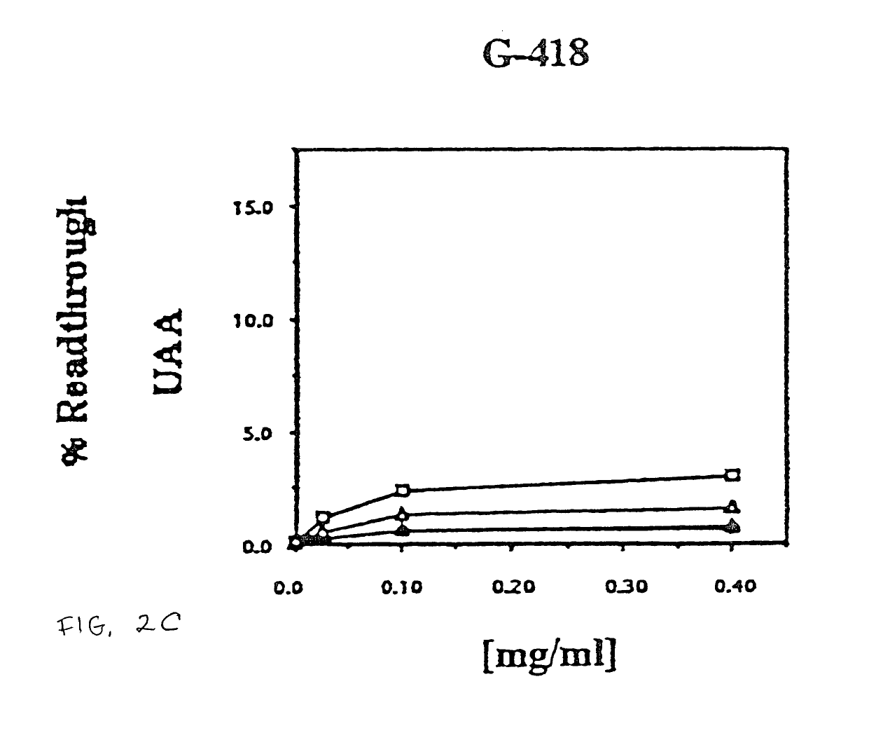

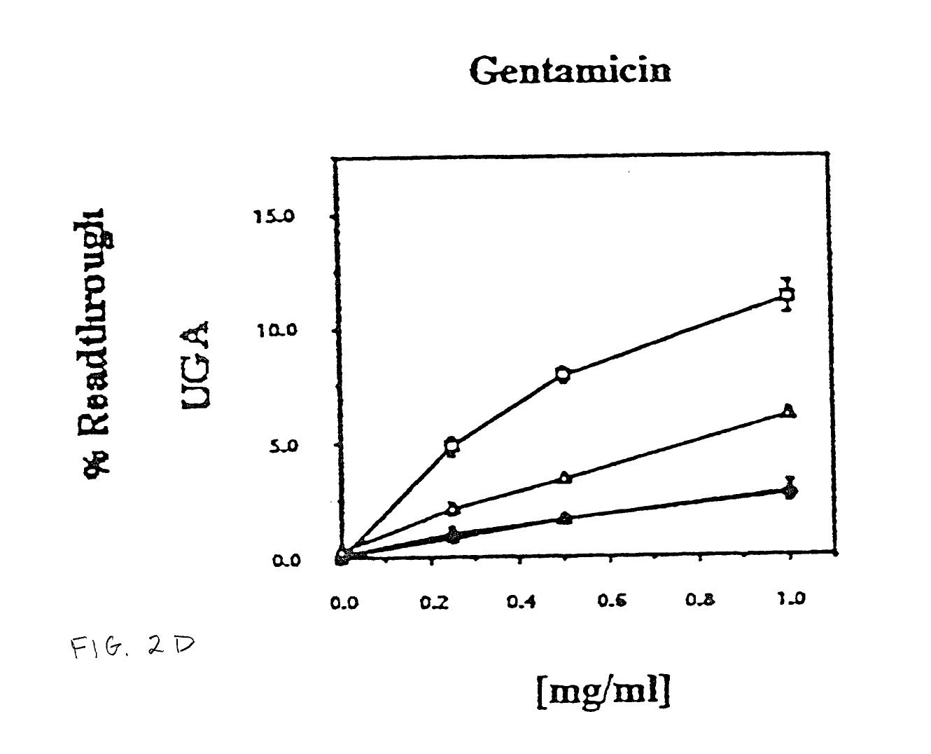

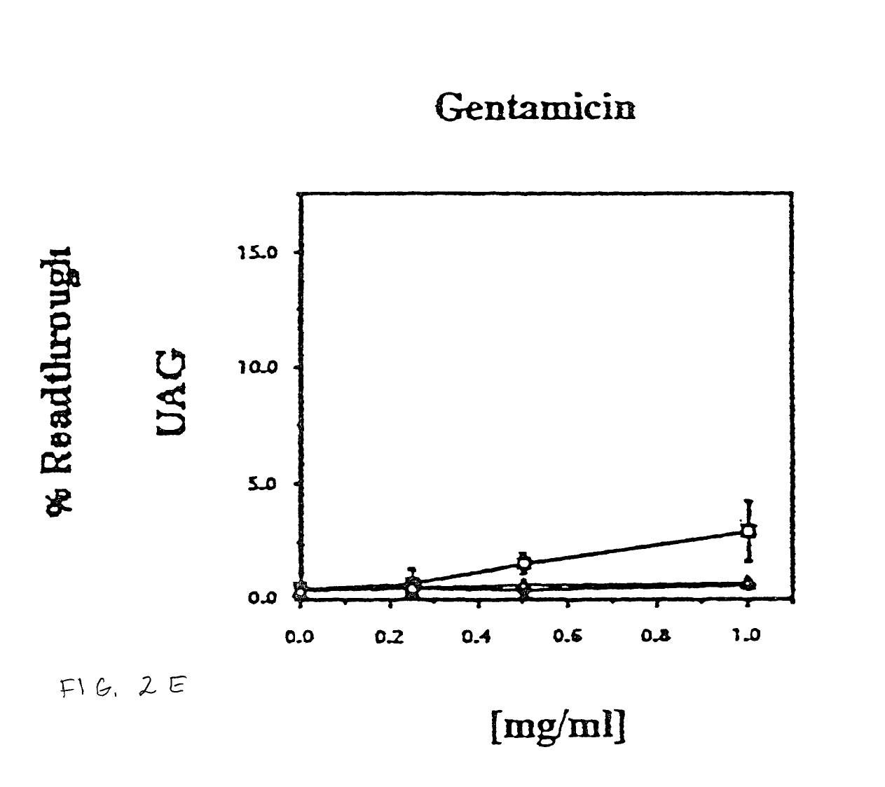

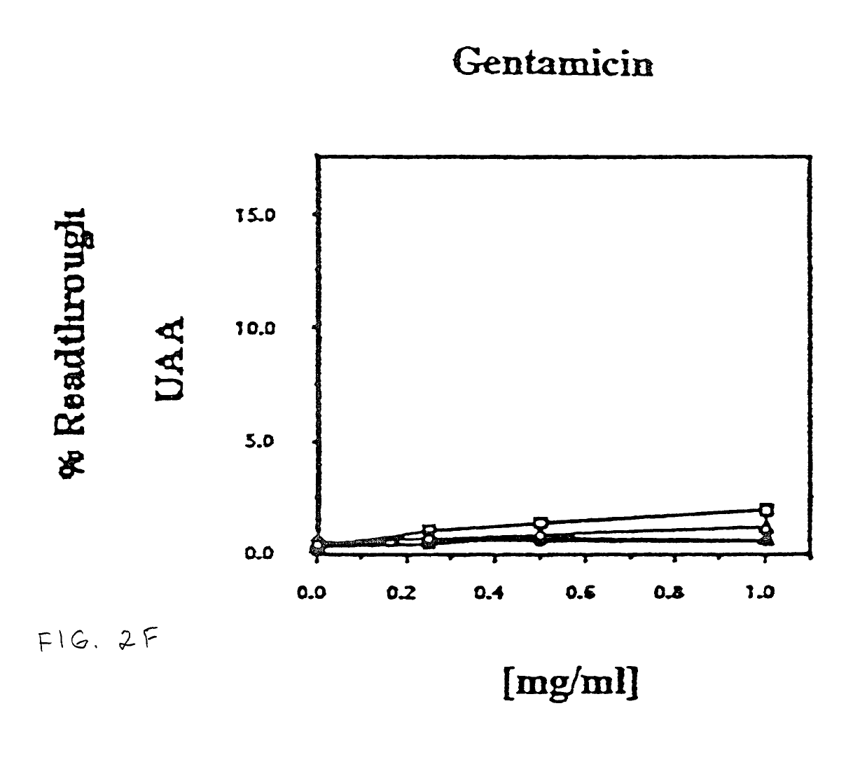

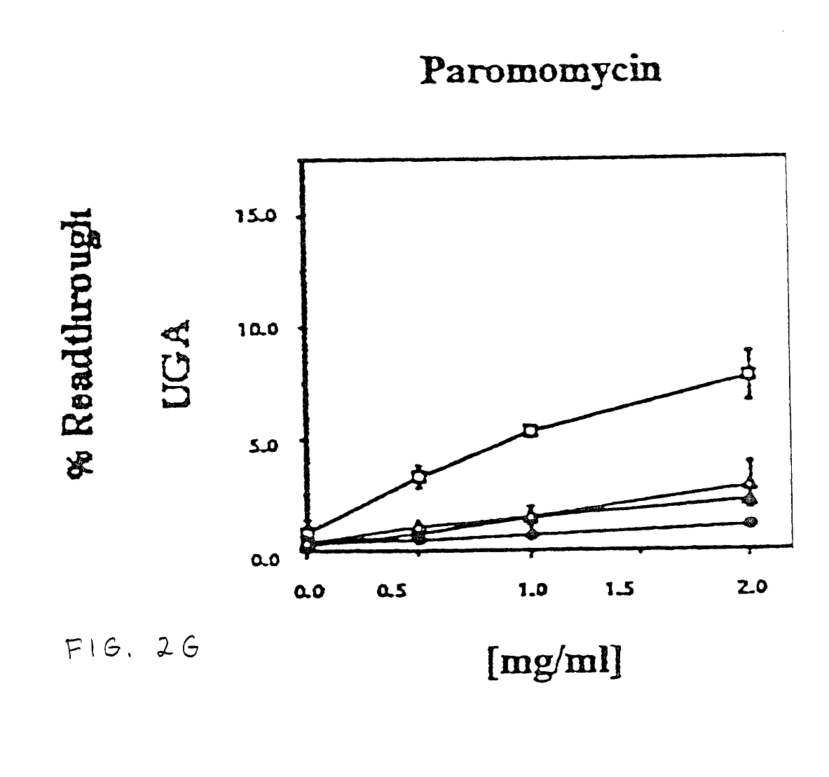

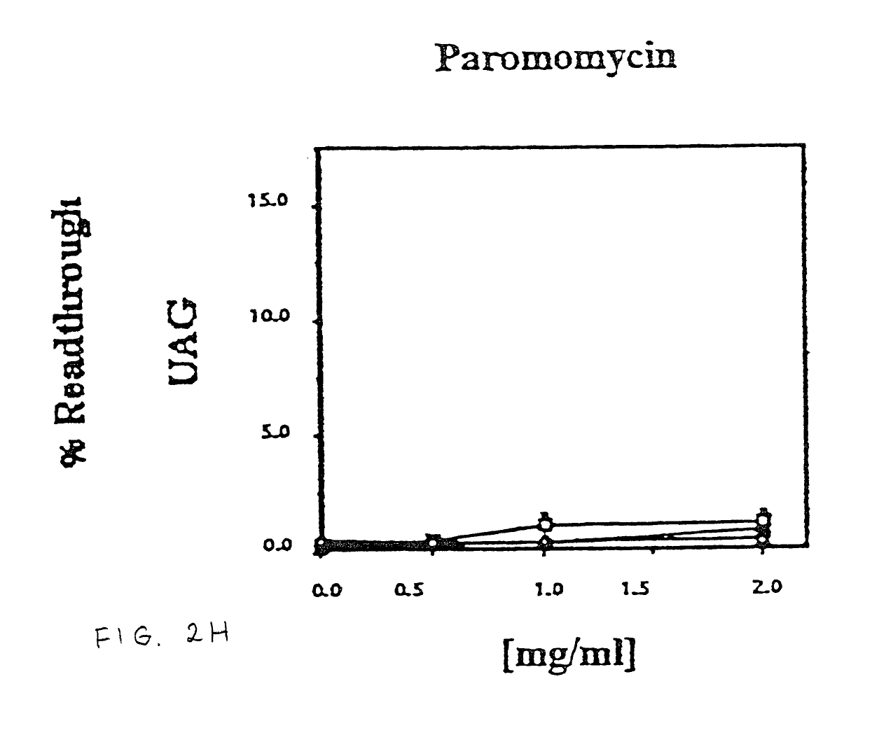

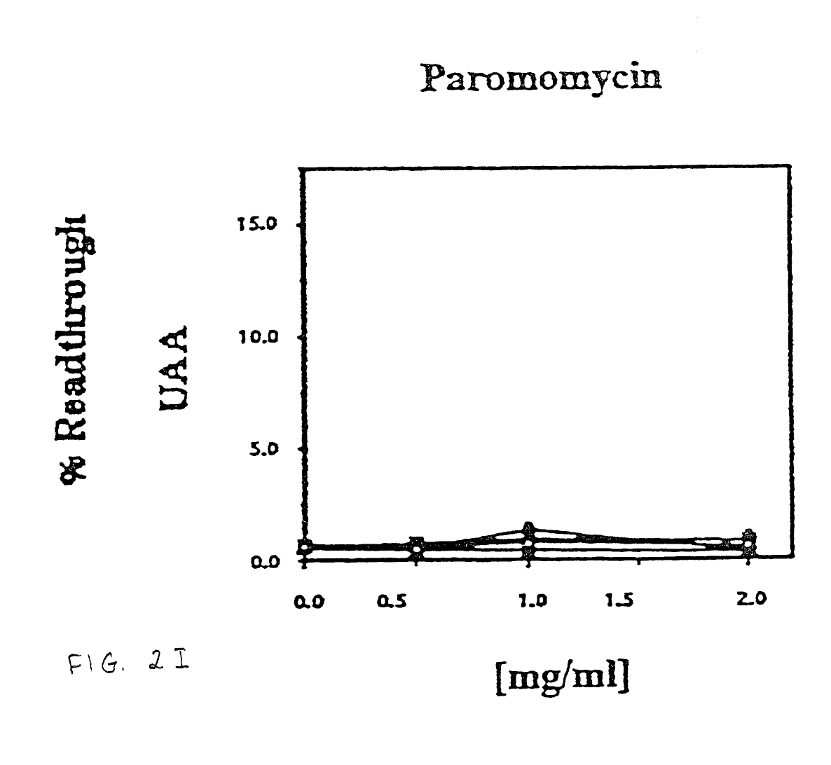

Referring now to FIGS. 2A-I, the results of aminoglycoside-induced stop codon readthrough are shown. The p2luc plasmids containing sequences encoding the stops codons UGA N, UAG N, and UAA N, where N is either A, C, G, or U, were tested in HEK293 cells for the ability of the aminoglycosides G-418, gentamicin, and paromomycin to induce stop codon readthrough. The efficiency of stop codon readthrough is shown on the y axis, and the concentration in milligrams (mg) per milliliter (ml) of aminoglycoside added to the growth media is shown on the x axis.

A significant difference in stop codon readthrough induced by the aminoglycosides was observed between UGA, UAG, and UAA. UGA showed the most translational readthrough in the presence of all three aminoglycosides tested, with UAG and UAA showing lower levels of readthrough. The nucleotide in the fourth position immediately after the stop codon also contributed to the frequency of stop codon readthrough. This effect is particularly obvious for UGA N, where N=C>U>A≧G in order of most to least stop codon readthrough. Of the three aminoglycosides tested, G-418 induced the most readthrough on a mass basis, followed by gentarnicin and paromomycin. Maximal levels of stop codon readthrough were observed at 0.4 mg/ml of G-418, lmg/ml of gentamicin, and 2 mg/ml of paromomycin. Inhibition of translation was monitored by examining the reduction in renilla luciferase activity in control transfections. The highest concentration of G-418 and gentamicin tested resulted in approximately a 50% and 20% reduction in overall translation, respectively, whereas paromomycin had little effect on translation at the concentrations tested.

The stop codon mutation UAA A is found in the dystrophin gene of the mdx mouse and has been shown, E. R. Barton-Davis et al., Aminoglycoside antibiotics restore dystrophin function to skeletal muscles of mdx mice, 104 J. Clin. Invest. 375-381 (1999), to be responsive to aminoglycoside treatment in vivo, resulting in approximately 10 to 20% accumulation of full-length dystrophin. Surprisingly, this combination of stop codon and 3′ nucleotide is a most efficient translational terminator and was one of the stop signals that showed the lowest efficiency of aminoglycoside-induced readthrough (FIGS. 2A-I). To address whether a larger sequence context surrounding the nonsense mutation in the dystrophin gene might lead to higher levels of readthrough, approximately 15 nucleotides upstream and downstream of the dystrophin mutation were tested in the dual luciferase assay system (SEQ ID NO: 25). Compared with that seen in the shorter UAA A constructs, no greater increase in aminoglycoside-induced translational readthrough was observed with this larger mdx sequence context in HEK293 cells. In addition, this sequence was tested alongside a UGA C control in the mouse myoblast C2C12 cell line (e.g., ATCC CRL-1772). C2C12 cells were grown as described above for HEK293 cells except that the C1C12 cells were maintained as a monolayer culture growing in Dulbecco's Modified Eagle Medium with 4,500 mg/L of D-glucose, L-glutamine, pyridoxine hydrochloride, and 110 mg/L of sodium pyruvate supplemented with 10% fetal bovine serum and 50 units/ml penicillin/50 μg/ml streptomycin. C2C12 cells were transfected as described above except that 0.075×10⁵ C2C12 cells were plated in each well. Aminoglycoside induced readthrough for the UGA C control was measured at 6% and less than 1% for the mdx premature stop codon. Although the C2C12 cells allowed readthrough at somewhat lower levels than HEK 293 cells for both control and mdx stop codons, these results confirm that aminoglycosides induced readthrough of the mdx premature stop codon at relatively low levels.

The tissue culture assays of the present invention show that the efficiency of stop codon readthrough in the presence of aminoglycosides is inversely proportional to the efficiency of translational termination. K. K. McCaughan et al., Translational termination efficiency in mammals is influenced by the base following the stop codon, 92 Proc. Nat'l Acad. Sci. U.S.A. 5431-5435 (1995), in the absence of these compounds.

A recent study of gentamicin-induced stop codon readthrough in the mdx mouse showed that approximately 10 to 20% of the full-length dystrophin protein was expressed on aminoglycoside treatment. E. R. Barton-Davis et al., Aminoglycoside antibiotics restore dystrophin function to skeletal muscles of mdx mice, 104 J. Clin. Invest. 375-381 (1999). This result is surprising in light of previous work and the present observations that UAA A, the nonsense mutation in the dystrophin gene of the mdx mouse, is one of the best translational terminators. W. P. Tate & S. A. Mannering, Three, four or more: the translational stop signal at length, 21 Mol. Microbiol. 213-219 (1996), and that readthrough is only slightly increased to approximately 1% readthrough by G-418, gentamicin, and paromomycin. It seems likely that other factors contribute to the relatively high levels of protein observed in the aminoglycoside-treated mdx mouse. There are several possible factors. First, a difference in translational termination may exist at UAA A between the mdx mouse and the tissue culture system used in this study or previous studies measuring termination efficiencies. In the hope of addressing this issue, the mdx sequence was tested in an established mouse myoblast cell line (C2C12 cells). No increase in gentamicin-induced readthrough was observed compared with that of the UAA A construct tested in HEK293 cells as shown in FIGS. 2A-I. Second, the stability of the dystrophin protein and the length of time over which the aminoglycoside treatments are administered may be important to obtaining high levels of protein. For example, the 10 to 20% levels of full length protein observed in the mdx mouse may result from the accumulation of protein over the course of treatment with only low-level translational readthrough occurring at any given time. It is believed that the half-life of wild-type dystrophin protein has not been determined; consequently, the influence of protein stability on dystrophin accumulation over the course of aminoglycoside treatment is hard to evaluate. Third, the treatment of the mdx mouse with aminoglycosides may induce elevated levels of dystrophin mRNA relative to untreated mice such that a low-level readthrough of the UAA stop codon results in a substantial increase in protein due to an increased number of messages being translated. Dystrophin MRNA levels in the untreated mdx mouse are approximately 20% of those found in wild-type mice, J. S. Chamberlain et al., Expression of the murine Duchenne muscular dystrophy gene in muscle and brain, 239 Science 1416-1418 (1988), suggesting that the presence of the premature stop codon results in degradation of these messages. This is likely due to the action of the nonsense-mediated decay pathway, which is known to preferentially degrade messages containing premature stop codons. X. Sun & L. E. Maquat, mRNA surveillance in mammalian cells: the relationship between introns and translation termination, 6 RNA 1-8 (2000); M. W. Hentze & A. E. Kulozik, A perfect message: RNA surveillance and nonsense-mediated decay, 96 Cell 307-310 (1999); P. Hilleren & R. Parker, mRNA surveillance in eukaryotes: kinetic proofreading of proper translation termination as assessed by mRNP domain organization?, 5 RNA 711-719 (1999). Low-level translational readthrough induced by aminoglycosides may be sufficient for the nonsense-containing dystrophin MRNA to avoid nonsense-mediated decay, resulting in elevated mRNA levels. In support of this notion, it has been demonstrated that translational readthrough of a UGA stop codon located in the coding region of the selenium-dependent glutathione peroxidase I gene by selenocysteine incorporation is sufficient to allow these messages to avoid nonsense-mediated decay. P. M. Moriarty et al., Selenium deficiency reduces the abundance of mRNA for Se-dependent glutathione peroxidase I by a UGA-dependent mechanism likely to be nonsense codon-mediated decay of cytoplasmic mRNA, 18 Mol. Cell Biol. 2932-2939 (1998).

Of these factors, the sequence context effect disclosed in the present invention should be generally applicable to many different diseases. The effects of mRNA levels and protein stability depend on the regulatory dynamics of a given gene and the turnover rate of its gene product; consequently, they are disease-specific. Direct analysis of dystrophin transcript levels and the protein stability in gentamicin-treated mdx mice and DMD patients is needed to evaluate the contribution of these factors to the amount of dystrophin protein produced. Despite the possible influence of these factors, the present invention demonstrates that the mdx mouse premature stop codon is the stop codon showing the least amount of aminoglycoside-induced readthrough, which suggests that the treatment of DMD by aminoglycosides may be even more effective than indicated by the mdx mouse study, because all other nonsense mutations should show an even greater response to treatment.

The suppression of genetic mutations by aminoglycosides or by other small molecules that affect the ribosome is not limited to stop codon readthrough. It is known in the art that frameshift mutations in certain sequence contexts are leaky as a result of ribosomal frameshifts during translation. R. B. Weiss RB et al., Ribosomal frameshifting from −2 to +50 nucleotides, 39 Prog. Nucleic Acid Res. Mol. Biol. 159-183 (1990). Limited studies in bacteria have indicated that aminoglycosides can enhance ribosomal frameshifting at low levels. C. P. van Buul et al., Increased translational fidelity caused by the antibiotic kasugamycin and ribosomal ambiguity in mutants harbouring the ksgA gene, 177 FEBS Lett. 119-124 (1984); J. F. Atkins et al., Low activity of P-galactosidase in frameshift mutants of Escherichia coli, 69 Proc. Nat'l Acad. Sci. U.S.A. 1192-1195 (1972).

In summary, the tissue culture assay of the present invention will help in investigating the parameters that determine the ability of drugs, such as aminoglycosides, to induce full-length protein from mutant messages. The present invention has direct implications for therapy in DMD as well as in many other diseases caused by premature stop codon mutations or other genetic sequence mutations, such as frameshift mutations. The present invention will help in the development of guidelines to predict how patients carrying such symptomatic mutations are likely to respond to aminoglycoside or other drug treatment. The tissue culture assay disclosed provides a method for examining the ability of drugs, such as aminoglycosides, to induce ribosomal frameshifts in addition to stop codon readthrough in mammalian cells and for screening for drugs with increased effectiveness. The tissue culture assay will also provide a tool for predicting response to drug (e.g., aminoglycoside) therapy based on the specific sequence of individual patients.

The subject matter claimed is:

1. A method of screening small-molecule compounds for ability to induce translational readthrough of a stop codon, comprising:
(a) providing a translational reporter vector comprising a cloning site disposed between a first coding sequence encoding a first luciferase and a second coding sequence encoding a second luciferase and inserting a test DNA comprising an in-frame stop codon at the cloning site to form a test vector such that the first and second coding sequences are in a same reading frame but are separated by the in-frame stop codon;
(b) inserting a control DNA in the translational reporter vector at the cloning site to form a control vector such that the first and second coding sequences are in the same reading frame and are not separated by a stop codon in such reading frame;
(c) separately transfecting aliquots of mammalian cells with the test vector and the control vector to result in transfected cells containing the test vector and transfected cells containing the control vector;
(d) separately incubating the transfected cells containing the test vector and the transfected cells containing the control vector in both the presence and absence of a small-molecule compound under conditions suitable for expression of the first luciferase and, if translational readthrough of the stop codon occurs, expression of the second luciferase;
(e) lysing the incubated cells and determining activities of the first luciferase and the second luciferase in the presence and the absence of the small-molecule compound; and
(f) calculating ratios of second luciferase activity to first luciferase activity in the presence and in the absence of the small-molecule compound, comparing such ratios, and determining that the small-molecule compound has induced translational readthrough of the stop codon when the ratio of second luciferase activity to first luciferase activity in the presence of the small-molecule compound exceeds the ratio of second luciferase activity to first luciferase activity in the absence of the small-molecule compound.

2. The method of claim 1 wherein the small-molecule compound comprises an aminoglycoside.

3. The method of claim 2 wherein the aminoglycoside is a member selected from the group consisting of streptomycin, gentamicin, tobramycin, kanamycin, neomycin, paromomycin, 10 G-418, and mixtures thereof.

4. The method of claim 2 wherein the aminoglycoside comprises gentamicin.

5. The method of claim 2 wherein the aminoglycoside comprises paromomycin.

6. The method of claim 2 wherein the aminoglycoside comprises G-418.

7. The method of claim 1 wherein the mammalian cells are human cells.

8. The method of claim 1 wherein the translational reporter vector comprises p2luc.

9. The method of claim 1 wherein the first luciferase comprises renilla luciferase and the second luciferase comprises firefly luciferase.

10. The method of claim 1 wherein the test DNA comprises at least a portion of a coding sequence of a gene that causes a genetic disease in an individual when the in-frame stop codon results in premature translational termination.

11. The method of claim 10 wherein the genetic disease is Duchenne muscular dystrophy.

12. The method of claim 11 wherein the test DNA comprises at least a portion of a coding sequence of a dystrophin gene.

13. A method of screening small-molecule compounds for ability to induce translational readthrough of a stop codon, comprising:
(a) providing a translational reporter vector comprising a cloning site disposed between a first coding sequence encoding a first reporter and a second coding sequence encoding a second reporter and inserting a test DNA comprising an in-frame stop codon at the cloning site to form a test vector such that the first and second coding sequences are in a same reading frame but separated by the in-frame stop codon;
(b) inserting a control DNA in the translational reporter vector at the cloning site to form a control vector such that the first and second coding sequences are in the same reading frame and are not separated by an in-frame stop codon;
(c) separately transfecting aliquots of cells with the test vector and the control vector to result in transfected cells containing the test vector and transfected cells containing the control vector;
(d) separately incubating the transfected cells containing the test vector and the transfected cells containing the control vector in both the presence and absence of a small-molecule compound under conditions suitable for expression of the first reporter and, if translational readthrough of the stop codon occurs, expression of the second reporter;
(e) lysing the incubated cells and determining activities of the first reporter and of the second report in both the presence and absence of the small-molecule compound; and
(f) calculating ratios of second reporter activity to first reporter activity in both the presence and absence of the small-molecule compound, comparing such ratios, and determining that the small-molecule compound has induced translational readthrough of the stop codon when the ratio of second reporter activity to first reporter activity in the presence of the small-molecule compound exceeds the ratio of second reporter activity to first reporter activity in the absence of the small-molecule compound.

14. The method of claim 13 wherein the small-molecule compound comprises an aminoglycoside.

15. The method of claim 14 wherein the aminoglycoside is a member selected from the group consisting of streptomycin, gentamicin, tobramycin, kanamycin, neomycin, paromomycin, G-418, and mixtures thereof.

16. The method of claim 14 wherein the aminoglycoside comprises gentamicin.

17. The method of claim 14 wherein the aminoglycoside comprises paromomycin.

18. The method of claim 14 wherein the aminoglycoside comprises G-418.

19. The method of claim 13 wherein the cells are mammalian cells.

20. The method of claim 19 wherein the mammalian cells are human cells.

21. The method of claim 13 wherein the translational reporter vector comprises p2luc.

22. The method of claim 13 wherein the first reporter comprises renilla luciferase and the second reporter comprises firefly luciferase.

23. The method of claim 13 wherein the test DNA comprises at least a portion of a coding sequence of a gene that causes a genetic disease in an individual when the in-frame stop codon causes premature translational termination.

24. The method of claim 23 wherein the genetic disease is Duchenne muscular dystrophy.

25. The method of claim 24 wherein the test DNA comprises at least a portion of a coding sequence of a dystrophin gene.

26. A method of screening drugs for potential for treating a genetic disease that is treatable by inducing translational readthrough of a stop codon causally linked with the genetic disease, comprising:
(a) providing a translational reporter vector comprising a cloning site disposed between a first coding sequence encoding a first reporter and a second coding sequence encoding a second reporter and inserting a test DNA comprising the stop codon causally linked with the genetic disease and flanking sequences thereof in the cloning site to form a test vector such that the first and second coding sequences are in a same reading frame but separated by the stop codon causally linked with the genetic disease, wherein such stop codon is in the same reading frame as the first and second coding sequences;
(b) inserting a control DNA in the translational reporter vector at the cloning site to form a control vector such that the first and second coding sequences are in the same reading frame and are not separated by a stop codon in the same reading frame as the first and second coding sequences;
(c) separately transfecting aliquots of cells with the test vector and the control vector to result in transfected cells containing the test vector and transfected cells containing the control vector;
(d) separately incubating the transfected cells containing the test vector and the transfected cells containing the control vector in both the presence and absence of a drug under conditions suitable for expression of the first reporter and, if translational readthrough of the stop codon occurs, expression of the second reporter;
(e) lysing the incubated cells and determining activities of the first reporter and of the second report in both the presence and absence of the drug; and
(f) calculating ratios of activity of the second reporter to activity of the first reporter both in the presence and the absence of the drug, comparing such ratios, and determining that the drug has induced translational readthrough of the stop codon, and thereby exhibits potential for treating the genetic disease, when the ratio of activity of the second reporter to activity of the first reporter in the presence of the drug exceeds the ratio of activity of the second reporter to activity of the first reporter in the absence of the drug.

27. The method of claim 26 wherein the drug comprises an aminoglycoside.

28. The method of claim 27 wherein the aminoglycoside is a member selected from the group consisting of streptomycin, gentamicin, tobramycin, kanamycin, neomycin, paromomycin, G-418, and mixtures thereof.

29. The method of claim 27 wherein the aminoglycoside comprises gentamicin.

30. The method of claim 27 wherein the aminoglycoside comprises paromomycin.

31. The method of claim 27 wherein the aminoglycoside comprises G-418.

32. The method of claim 26 wherein the cells are mammalian cells.

33. The method of claim 32 wherein the mammalian cells are human cells.

34. The method of claim 26 wherein the translational reporter vector comprises p2luc.

35. The method of claim 26 wherein the first reporter comprises renilla luciferase and the second reporter comprises firefly luciferase.

36. The method of claim 26 wherein the genetic disease is Duchenne muscular dystrophy.

37. The method of claim 36 wherein the test DNA comprises at least a portion of a coding sequence of a dystrophin gene.

38. A method of screening drugs for potential for treating Duchenne muscular dystrophy caused by premature translational termination of dystrophin caused by a mutation that introduces a premature stop codon into a coding sequence of dystrophin, comprising:
(a) providing a translational reporter vector comprising a cloning site disposed between a first coding sequence encoding a first reporter and a second coding sequence encoding a second reporter and inserting a test DNA comprising at least a portion the coding sequence of dystrophin comprising the premature stop codon to form a test vector such that the first and second coding sequences are in a same reading frame but are separated by the premature stop codon, wherein such premature stop codon is in the same reading frame as the first and second coding sequences;
(b) inserting a control DNA in the translational reporter vector at the cloning site to form a control vector such that the first and second coding sequences are in the same reading frame and are not separated by a stop codon in such reading frame;
(c) separately transfecting aliquots of cells with the test vector and the control vector to result in transfected cells containing the test vector and transfected cells containing the control vector;
(d) separately incubating the transfected cells containing the test vector and the transfected cells containing the control vector in both the presence and absence of a drug under conditions suitable for expression of the first reporter and, if translational readthrough of the premature stop codon occurs, expression of the second reporter;
(e) lysing the incubated cells and determining activities of the first reporter and of the second reporter both in the presence and in the absence of the drug; and
(f) calculating ratios of the activity of the second reporter to the activity of the first reporter both in the presence and in the absence of the drug, comparing such ratios, and determining that the drug has induced translational readthrough of the premature stop codon, and thereby exhibits potential for treating Duchenne muscular dystrophy, when the ratio of activity of the second reporter to the activity of the first reporter in the presence of the drug exceeds the ratio of the activity of the second reporter to the activity of the first reporter in the absence of the drug.

39. The method of claim 38 wherein the drug comprises an aminoglycoside.

40. The method of claim 39 wherein the aminoglycoside is a member selected from the group consisting of streptomycin, gentamicin, tobramycin, kanamycin, neomycin, paromomycin, G-418, and mixtures thereof.

41. The method of claim 39 wherein the aminoglycoside comprises gentamicin.

42. The method of claim 39 wherein the aminoglycoside comprises paromomycin.

43. The method of claim 39 wherein the aminoglycoside comprises G-418.

44. The method of claim 38 wherein the cells are mammalian cells.

45. The method of claim 44 wherein the mammalian cells are human cells.

46. The method of claim 38 wherein the translational reporter vector comprises p2luc.

47. The method of claim 38 wherein the first reporter comprises renilla luciferase and the second reporter comprises firefly luciferase.

48. A method for predicting whether a patient afflicted with a genetic disease caused by a mutation creating a premature stop codon will be effectively treated for such genetic disease with a selected drug comprising:
(a) providing a translational reporter vector comprising a cloning site disposed between a first coding sequence encoding a first reporter and a second coding sequence encoding a second reporter and inserting a test DNA derived from the patient comprising the premature stop codon and flanking sequences thereof in the cloning site to form a test vector such that the first and second coding sequences are in a same reading frame but separated by the premature stop codon in such same reading frame;
(b) inserting a control DNA in the translational reporter vector at the cloning site to form a control vector such that the first and second coding sequences are in the same reading frame and are not separated by a stop codon in the same reading frame as the first and second coding sequences;
(c) separately transfecting aliquots of cells with the test vector and the control vector to result in transfected cells containing the test vector and transfected cells containing the control vector;
(d) separately incubating the transfected cells containing the test vector and the transfected cells containing the control vector in both the presence and absence of the selected drug under conditions suitable for expression of the first reporter and, if translational readthrough of the premature stop codon occurs, expression of the second reporter;
(e) lysing the incubated cells and determining activities of the first reporter and of the second report in both the presence and absence of the selected drug; and
(f) calculating ratios of activity of the second reporter to activity of the first reporter both in the presence and the absence of the selected drug, comparing such ratios, and determining that the selected drug has induced translational readthrough of the premature stop codon, and thereby the patient can be effectively treated for such genetic diseases, when the ratio of activity of the second reporter to activity of the first reporter in the presence of the selected drug exceeds the ratio of activity of the second reporter to activity of the first reporter in the absence of the selected drug.

49. The method of claim 48 wherein the drug comprises an aminoglycoside.

50. The method of claim 49 wherein the aminoglycoside is a member selected from the group consisting of streptomycin, gentamicin, tobramycin, kanamycin, neomycin, paromomycin, G-418, and mixtures thereof.

51. The method of claim 49 wherein the aminoglycoside comprises gentamicin.

52. The method of claim 49 wherein the aminoglycoside comprises paromomycin.

53. The method of claim 49 wherein the aminoglycoside comprises G-418.

54. The method of claim 48 wherein the cells are mammalian cells.

55. The method of claim 54 wherein the mammalian cells are human cells.

56. The method of claim 48 wherein the translational reporter vector comprises p2luc.

57. The method of claim 48 wherein the first reporter comprises renilla luciferase and the second reporter comprises firefly luciferase.

58. The method of claim 48 wherein the genetic disease is Duchenne muscular dystrophy.

59. The method of claim 58 wherein the test DNA comprises at least a portion of a coding sequence of a dystrophin gene.

60. A method for screening drugs for ability to induce translational recoding at frameshift mutations comprising:
(a) providing a translational reporter vector comprising a cloning site disposed between a first coding sequence encoding a first reporter and a second coding sequence encoding a second reporter wherein the first and second coding sequences are in different reading frames and inserting a test DNA in the translational reporter vector at the cloning site to form a test vector such that the first and second coding sequences remain in different reading frames;
(b) inserting the test DNA in the translation reporter vector at the cloning site to form a control vector such that the first and second coding sequences are in a same reading frame;
(c) separately transfecting aliquots of cells with the test vector and the control vector to result in transfected cells containing the test vector and transfected cells containing the control vector;
(d) incubating the transfected cells containing the test vector and the transfected cells containing the control vector in the presence and in the absence of a selected drug under conditions suitable for expression of the first reporter and, if translational recoding of the frameshift mutation occurs, expression of the second reporter;
(e) lysing the transfected cells containing the test vector and the transfected cells containing the control vector and determining activities of the first reporter and the second reporter both in the presence and in the absence of the selected drug; and
(f) calculating ratios of the activity of the second reporter to the activity of the first reporter both in the presence and in the absence of the drug, comparing such ratios, and determining that the drug has induced translational recoding of the frameshift mutation when the ratio of activity of the second reporter to the activity of the first reporter in the presence of the selected drug exceeds the ratio of the activity of the second reporter to the activity of the first reporter in the absence of the selected drug.

61. The method of claim 60 wherein the cells are mammalian cells.

62. The method of claim 61 wherein the mammalian cells are human cells.

63. The method of claim 60 wherein the translational reporter vector comprises p2luc.

64. The method of claim 60 wherein the first reporter comprises renilla luciferase and the second reporter comprises firefly luciferase.

65. A method for predicting whether a patient afflicted with a genetic disease caused by a mutation creating a frameshift will be effectively treated for such genetic disease with a selected drug comprising:
(a) providing a translational reporter vector comprising a cloning site disposed between a first coding sequence encoding a first reporter and a second coding sequence encoding a second reporter and inserting a test DNA derived from the patient comprising the frameshift and flanking sequences thereof in the cloning site to form a test vector such that the first and second coding sequences are in different reading frames;
(b) inserting the test DNA in the translational reporter vector at the cloning site to form a control vector such that the first and second coding sequences are in a same reading frame;
(c) separately transfecting aliquots of cells with the test vector and the control vector to result in transfected cells containing the test vector and transfected cells containing the control vector;
(d) separately incubating the transfected cells containing the test vector and the transfected cells containing the control vector in both the presence and absence of the selected drug under conditions suitable for expression of the first reporter and, if translational recoding of the frameshift occurs, expression of the second reporter;
(e) lysing the incubated cells and determining activities of the first reporter and of the second report in both the presence and absence of the selected drug; and
(f) calculating ratios of activity of the second reporter to activity of the first reporter both in the presence and the absence of the selected drug, comparing such ratios, and determining that the selected drug has induced recoding of the frameshift, and thereby the patient can be effectively treated for such genetic diseases, when the ratio of activity of the second reporter to activity of the first reporter in the presence of the selected drug exceeds the ratio of activity of the second reporter to activity of the first reporter in the absence of the selected drug.

66. The method of claim 65 wherein the cells are mammalian cells.

67. The method of claim 66 wherein the mammalian cells are human cells.

68. The method of claim 65 wherein the translational reporter vector comprises p2luc.

69. The method of claim 65 wherein the first reporter comprises renilla luciferase and the second reporter comprises firefly luciferase.
